SEQUENCE LISTING (2 sequences)

SEQ ID NO: 1 - DNA, 1362 bp, synthetic construct
  source 1..1362: mol_type = other DNA
atgagcgtcaacaacccgcaaacccgtgaatggcaaaccctgagcggggagcatcacctcgcacccttcagtgactacaagcaactgaaggagaaggggccgcgcatcatcaccaaggcccagggtgtgcatttgtgggatagcgaggggcacaagatcctcgacggcatggccggtctatggtgcgtggcggtcggctacggccgtgaagagctggtgcaggcggcagaaaaacagatgcgcgagctgccgtactacaacctgttcttccagaccgctcacccgccggcgctcgagctggccaaagcgatcaccgaagtggcgccgaaaggtatgacccatgtgttcttcaccggctccggctccgaaggcaacgacactgtgctgcgcatggtgcgtcactactgggcgcttaagggcaaaccgcacaagcagaccatcatcggccgtatcaacggttaccacggttccaccttcgccggtgcttgcctgggtggcatgagcggcatgcacgagcagggtggcttgccgatcccgggtatcgttcacatccctcagccttactggttcggcgagggcggcgacatgacccctgacgaattcggtgtctgggctgccgagcagctggagaagaagatcctcgaagtcggcgaagacaacgtcgctgccttcatcgccgagccgatccagggcgcaggtggcgtgatcatcccgccggaaacctactggcccaaggtgaaggagatccttgccaagtacgacatcctgttcgtcgccgacgaggtgatctgcggcttcggccgtaccggcgagtggttcggctctgactactacgacctcaagcccgacctgatgaccatcgcgaaaggcctgacctctggttacatccccatgggcggtgtgatcgtgcgtgacaccgtggccaaggtgatcagcgaaggcggcgacttcaatcacggtttcacctactccggccacccggtggcggccgcggtgggcctggaaaacctgcgcatcctgcgtgacgagaaaattgtcgagaaggcgcgcacggatacggcaccgtatttgcaaaagcgtttgcgcgagctgcaggaccatcctctggtgggtgaagtgcgcggcctgggcatgctgggtgcgatcgagctggtcaaggacaaggccacccgcagccgttacgaaggcaaaggcgtgggcatgatctgtcgcaccttctgctttgagaacggcctgatcatgcgtgcggtgggtgacaccatgatcatcgcgccgccgctggtaatcagccatgcggagatcgacgaactggtggaaaaggcacgcaagtgcctggatcttacccttgaggcgattcgataa

SEQ ID NO: 2 - protein, 453 aa, synthetic construct
MSVNNPQTREWQTLSGEHHLAPFSDYKQLKEKGPRIITKAQGVHLWDSEGHKILDGMAGLWCVAVGYGREELVQAAEKQMRELPYYNLFFQTAHPPALELAKAITEVAPKGMTHVFFTGSGSEGNDTVLRMVRHYWALKGKPHKQTIIGRINGYHGSTFAGACLGGMSGMHEQGGLPIPGIVHIPQPYWFGEGGDMTPDEFGVWAAEQLEKKILEVGEDNVAAFIAEPIQGAGGVIIPPETYWPKVKEILAKYDILFVADEVICGFGRTGEWFGSDYYDLKPDLMTIAKGLTSGYIPMGGVIVRDTVAKVISEGGDFNHGFTYSGHPVAAAVGLENLRILRDEKIVEKARTDTAPYLQKRLRELQDHPLVGEVRGLGMLGAIELVKDKATRSRYEGKGVGMICRTFCFENGLIMRAVGDTMIIAPPLVISHAEIDELVEKARKCLDLTLEAIR

(19) United States
(12) Patent Application Publication
(10) Pub. No.: US 2025/0236851 A1
(43) Pub. Date: Jul. 24, 2025

(54) Pseudomonas Putida omega-Transaminase Gene with Broad Substrate Spectrum and Application Thereof

(71) Applicant: SHANXI UNIVERSITY, Taiyuan (CN)

(72) Inventors: Yawei SHI, Taiyuan (CN); Jing LU, Taiyuan (CN); Shuai LI, Taiyuan (CN); Lei WANG, Taiyuan (CN); Yuting LIU, Taiyuan (CN)

(21) Appl. No.: 19/013,153

(22) Filed: Jan. 8, 2025

(30) Foreign Application Priority Data
Jan. 22, 2024 (CN) 202410088201.5

(51) Int. Cl. C12N 9/10

(52) U.S. Cl. CPC C12N 9/1096; C12Y 206/01018

(57) ABSTRACT

A Pseudomonas putida ω-aminotransferase gene with broad substrate spectrum and an application thereof are provided. According to the disclosure, codon optimization is carried out on an ω-transaminase gene, and the nucleotide sequence of the ω-transaminase gene is shown in SEQ ID NO: 1. The crude enzyme solution, crude enzyme powder and pure enzyme of ω-aminotransferase prepared from ω-aminotransferase gene can be used as an enzyme preparation to catalyze the transamination reaction of substrates to prepare chiral amino alcohols and chiral amines. The substrate spectrum of the recombinant ω-transaminase obtained by the disclosure is broad, and 15 kinds of chiral amino alcohols and chiral amines with high added value are asymmetrically catalyzed and synthesized; in particular, 4-hydroxy-2-butanone is catalyzed to produce pharmaceutical intermediate (R)-3-amino-1-butanol.

### CROSS-REFERENCE TO RELATED APPLICATIONS

This application claims priority to Chinese Patent Application No. 202410088201. 5, filed on Jan. 22, 2024, the contents of which are hereby incorporated by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:

File name: US-2024-18000-SequenceListing

Creation date: 3 Jan. 2025

Byte size: 4479

### TECHNICAL FIELD

The disclosure belongs to enzyme engineering and genetic engineering, and particularly relates to a Pseudomonas putida ω-transaminase gene with broad substrate spectrum and an application thereof.

### BACKGROUND

Two compounds that are mirror images of each other and cannot overlap are called enantiomers or optical isomers, because these isomers have chiral factors and play an important role in pharmaceutical field, metabolic process and toxicological properties, so they are called chiral drugs. The lower the effective dose of a drug, the greater the difference between its enantiomer and pharmacology. However, with the development of society and the pursuit of human health, the demand for chiral drugs with high activity is in short supply. Because chiral drugs are widely used in the field of antibiotics, it is difficult for chiral drugs to be approved by the pharmaceutical administration, and there are few chiral drugs naturally existing, which makes the preparation of chiral drugs particularly important. The physiological activity and pharmacological action of chiral drugs have become an important field in pharmaceutical research.

Enzymatic asymmetric reaction is an important method to synthesize chiral amine drugs because of its mild reaction conditions, high selectivity and environmental friendliness. Synthesis of chiral amines by enzymatic asymmetric synthesis method can be divided into reductive amination, hydroamination reaction and transamination reaction. Enzymes involved in asymmetric synthesis of chiral amines include ketoimine reductase, L-leucine dehydrogenase, phenylalanine lyase and ω-transaminase. Among them, asymmetric synthesis of chiral amines by ω-transaminase has the advantages of low substrate cost, high product purity and environmental friendliness, which has attracted more and more attention from researchers at home and abroad. ω-transaminase is a kind of aminotransaminase that is dependent on coenzyme pyridoxal phosphate, which has a wide range of acceptor or donor molecular spectrum and has great potential in synthesizing a variety of optically pure amine small molecules.

AIDS, also known as acquired immunodeficiency syndrome, is a very harmful infectious disease caused by human immunodeficiency virus (HIV). Anti-retroviral drugs have made AIDS a chronic disease. At present, HIV integrase inhibitors are effective drugs to treat AIDS, and (R)-3-amino-1-butanol is an important intermediate in the synthesis of HIV integrase inhibitors. At present, there are published literatures disclosing that (R)-3-amino-1-butanol is prepared by biocatalysis where 4-hydroxy-2-butanone is subjected to asymmetric reductive amination by selective transaminase in Aspergillus terreus to produce (R)-3-amino-1-butanol. However, the conversion rate is only 5%. In present disclosure, 4-hydroxy-2-butanone is asymmetrically synthesized into (R)-3-amino-1-butanol by utilizing the catalytic ability of ω-transaminase, which provides a new scheme for biocatalytic synthesis of (R)-3-amino-1-butanol.

### SUMMARY

Aiming at the technical problems, the present disclosure provides a Pseudomonas putida ω-transaminase gene with broad substrate spectrum as a catalyst to synthesize various chiral amino alcohols and chiral amines with high added value.

In order to achieve the above objective, the present disclosure provides following technical scheme.

A Pseudomonas putida ω-transaminase gene with broad substrate spectrum is provided, with a nucleotide sequence shown in SEQ ID NO:1.

A protein ω-transaminase obtained by translating the ω-transaminase gene as described above is provided, and the amino acid sequence of the ω-transaminase is shown in SEQ ID NO:2.

A recombinant expression vector including Pseudomonas putida ω-transaminase as described above is provided.

Further, the recombinant expression vector is any one of pET-28a, pET-29a, pET-30a, pET-32a, pCold I and pCold II.

A recombinant host cell including the recombinant expression vector as described above is provided.

Further, the host cell is any one of BL21 (DE3), BL21 Star (DE3), BL21 (DE3) pLysS, C41 (DE3), C43 (DE3), BL21 (DE3) pLysE, Origami series, AD494 (DE3), BL21Trxb (DE3), and SHuffle T7.

An enzyme preparation including protein ω-transaminase as described above is provided.

An application of the ω-transaminase described above in preparing chiral amino alcohols and chiral amines by catalyzing transamination of substrate;

The substrate includes one of or more of methyl isobutyl ketone, propanal, 2-heptanone, pyruvic acid, 5-methyl-2-hexanone, cyclohexanone, 4-hydroxy-2-butanone, hexanal, acetaldehyde, hydroxyacetone, diaectone alcohol, 2-hexanone, α-ketoglutaric acid, glyoxylic acid, n-butyraldehyde.

Further, the method for preparing chiral amino alcohols and chiral amines is as follows:

catalyzing amino donor and amino acceptor by the enzyme preparation and coenzyme pyridoxal phosphate as described above to generate chiral amino alcohols under the reaction temperature of 35° C. and 100 mM of triethanolamine buffer solution with pH of 8.5.

Compared with the prior art, the disclosure has the following beneficial effects.

Firstly, the crude enzyme solution, crude enzyme powder and pure enzyme of ω-aminotransferase prepared by ω-aminotransferase gene of the present disclosure can be used as enzyme preparation to catalyze transamination reaction of substrates to prepare chiral amino alcohols and chiral amines.

Secondly, the recombinant transaminase catalyst obtained by the disclosure can asymmetrically catalyze the synthesis of 15 kinds of chiral amino alcohols and chiral amines with high added value, and especially the specific activity for 4-hydroxy-2-butanone reaches 2397 U/g, which is much higher than the reported 596 U/g.

Lastly, the recombinant ω-transaminase obtained by the disclosure has a broad spectrum of substrates, and especially it can catalyze 4-hydroxy-2-butanone to generate a pharmaceutical intermediate (R)-3-amino-1-butanol which can be used as an intermediate for preparing compounds with HIV integrase inhibitory activity.

### BRIEF DESCRIPTION OF THE DRAWINGS

FIG. 1 shows a recombinant plasmid map of ω-aminotransferase TA.

FIG. 2 shows the electrophoresis diagram of protein purification of recombinant ω-aminotransferase TA, in which lane 1 is the protein standard marker, lane 2 is the broken whole bacteria sample, lane 3 is the supernatant sample after centrifugation, lane 4 is the precipitated sample after centrifugation, lane 5 is the penetration sample, lane 6 was the sample of affinity purification, and lane 7 is the purified protein of ω-transaminase TA.

FIG. 3 shows a substrate spectrum of the catalytic reaction of recombinant ω-transaminase.

FIG. 4 shows spectrums of corresponding product generated by catalyzing the different substrates as shown in FIG. 3 by ω-transaminase.

FIG. 5 shows chemical reaction equation of (R)-3-amino-1-butanol catalyzed by recombinant ω-TA.

FIG. 6A shows the retention time of standard samples acetophenone and (R)-3-amino-1-butanol detected by High Performance Liquid Chromatography (HPLC).

FIG. 6B shows retention time of the product detected by HPLC.

FIG. 7 shows the relative activity of (R)-3-amino-1-butanol catalyzed by recombinant ω-TA at different temperatures.

FIG. 8 shows the relative activity of (R)-3-amino-1-butanol catalyzed by recombinant ω-TA in different pH and different kinds of buffers.

### DETAILED DESCRIPTION OF THE EMBODIMENTS

In order to facilitate the understanding of the present disclosure, the present disclosure will be described more fully below. However, the present disclosure can be embodied in many different forms and is not limited to the embodiments described herein. On the contrary, these embodiments are provided to make the disclosure of the present disclosure more thorough and comprehensive.

### Embodiment 1

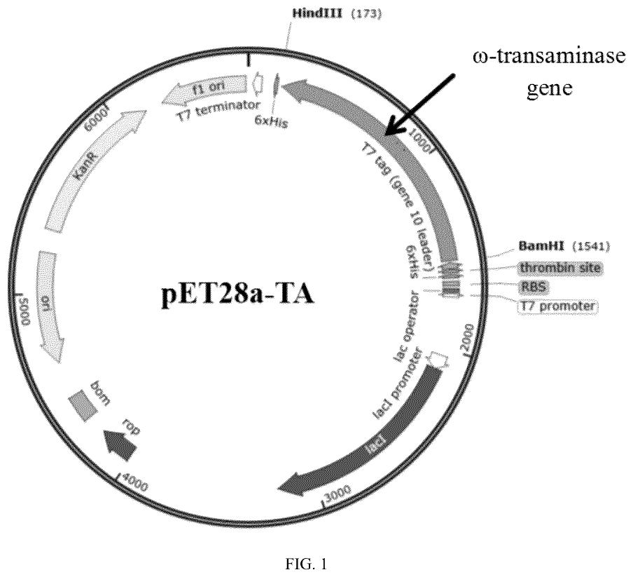

Cloning of ω-transaminase Gene and Construction of Recombinant Vector
(1) analyzing the whole genome sequence of a novel Pseudomonas putida to find out a gene encoding ω-transaminase, designing the ω-transaminase artificially, and adding His-tag to the initial coding position of ω-transaminase protein sequence for subsequent protein purification;
(2) in this embodiment, selecting Escherichia coli expression vector pET-28a (including multiple cloning sites HindIII and BamHI) and host cell BL21 (DE3) for the description of recombinant construction (havig same effect as selecting any one of Escherichia coli expression vectors pET-28a, pET-29a, pET-30a, pET-32a, pCold I and pCold II containing multiple cloning sites NdeI, SacI, XhoI, NcoI, EcoRI, SalI, and any one of Escherichia coli expression host cells BL21 (DE3), BL21 Star (DE3), BL21 (DE3) pLysS, C41 (DE3), C43 (DE3), BL21 (DE3) pLysE, Origami series, AD494 (DE3), BL21 Trxb (DE3) and SHuffle T7.
(3) designing HindIII and BamHI restriction sites at both ends of the original gene sequence obtained in step (1), and cloning the nucleotide sequence encoding ω-transaminase by polymerase chain reaction (PCR) technology, as shown in SEQ ID NO:1;
(4) digesting the genes obtained in step (3) and pET-28a by endonucleases HindIII and BamHI, respectively, and recovering the digested gene fragments by nucleic acid electrophoresis (0.1% agarose) and a kit (SanPrep column DNA gel recovery kit, Sangon Biotech (Shanghai) Co., Ltd.), and then ligating digested target gene fragments to the digested pET-28a;
(5) preparing a 10 μL of ligation system including 1 μL of 10 xT4 DNA ligation Buffer (Takara Company), 6 μL of target gene fragment, 2 μL of plasmid fragment and 1 μL of T4 DNA ligase, and performing the ligation is at 16° C. overnight; and
(6) after completing the ligation, transforming the reaction solution into DH5a competent cells, and the next day, selecting a single clone for sequencing verification, and extracting a recombinant plasmid with correct sequencing to obtain a recombinant expression vector containing the transaminase coding gene, and naming the recombinant expression vector pET28a-TA. The recombinant plasmid profile is shown in FIG. 1.

### Embodiment 2

Construction of Genetically Engineered Bacteria of ω-aminotransferase and Preparation of Cell Catalyst
(1) transforming the recombinant expression vector pET28a-TA prepared in Embodiment 1 into competent E. coli BL21 (DE3) by thermal shock method, coating the transformed product on a lysogeny broth (LB) plate containing kanamycin (50 μg/mL), and then culturing overnight at 37° C., and then selecting single colonies to be transferred to LB liquid medium containing 50 μg/mL kanamycin, and culturing at 37° C. for 12 hours, followed by gene sequencing. The genetic engineering bacteria containing the recombinant expression vector pET28a-TA is named as E. coli BL21 (DE3) pET28a-TA;
(2) inoculating the genetically engineered bacteria containing the coding sequence of ω-transaminase into 5 mL LB liquid culture medium (including kanamycin with final concentration of 50 μg/mL, 10 g/L of peptone, 5 g/L of yeast extract, 10 g/L of sodium chloride, and deionized water as the solvent, pH 7.0), putting the genetically engineered bacteria in a shaker, and cultivating at 37° C. and 200 r/min for 10-12 hours to obtain seed liquid; and
(3) transferring the seed liquid to a shake flask (final concentration of kanamycin 50 μg/mL) containing LB culture medium (100 mL) in a super-clean workbench, put it in a shaking table, and culturing it for 2-3 hours at 37° C. and 200 r/min; when the OD₆₀₀ value of the culture solution reaches 0.6-0.8, adding IPTG with final concentration of 0.1-0.8 mM to induce expression under induction temperature of 16-37° C. (the preferred IPTG concentration in this embodiment is 0.2 mM, and the preferred induction temperature is 16° C.); after 24 hours of induction, collecting cells by centrifugation to obtain recombinant cells containing ω-aminotransferase.

### Embodiment 3

### Purification of ω-Aminotransferase Protein

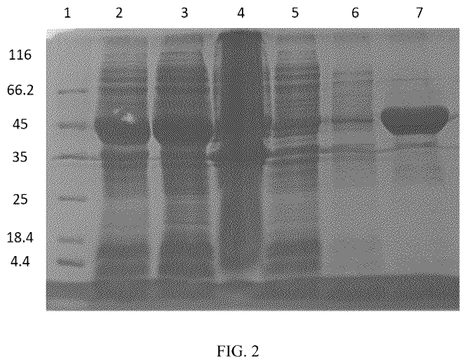

(1) weighing 10 g of the recombinant wet cells of ω-aminotransferase obtained in Embodiment 2 and adding them into 100 mL of buffer A (25 mM Tris HCl, pH 8.0; 300 mM NaCl), melting at room temperature, and then using an ultrasonic crusher to crush cells, where the horn type is q 10, the ultrasonic power is 100 W, the ultrasonic is turned on for 4 seconds, and the ultrasonic is turned off for 6 seconds. During the crushing process, the temperature is reduced with a low-temperature circulating device, and the ultrasonic crushing is carried out for 15 minutes; the collected crushing liquid is centrifuged, and the supernatant is collected;
(2) assembling a protein purification device, a self-assembled nickel column (10 mL Sepharose 6Fast Flow, GE) and a collection device; passing the collected supernatant through a 0.22 μm filter membrane, then adding it into the self-assembled nickel column which has been balanced for combination at 4° C. for 0. 5 hour, then collecting the permeate in the nickel column, and then using a washing buffer (25 mM Tris HCl, pH 8.0; 300 mM NaCl; 20 mM imidazole) to wash away impurity protein in the nickel column, and finally adding 15 mL of elution buffer (25 mM Tris HCl, pH 8.0; 300 mM NaCl; 500 mM imidazole), standing for 10 minutes and collecting eluent;
(3) filtering the collected eluent through a 0.22 μm filter membrane, and then loading the sample on Superdex200 gel column which is balanced by a buffer solution (25 mM Tris HCl, pH 8.0; 300 mM NaCl), collecting protein samples, using a 10-30 kDa ultrafiltration tube for centrifugal concentration at 4° C. and 4,000 rpm, determining the concentration of pure protein by Coomassie Brilliant Blue (CBB method) after concentration, putting it in a refrigerator at 80° C. after quick freezing in liquid nitrogen, and analyzing the purity of the target protein by SDS-PAGE. The results are shown in FIG. 2.

### Embodiment 4

### Determination of ω-Transaminase Activity and Specific Activity

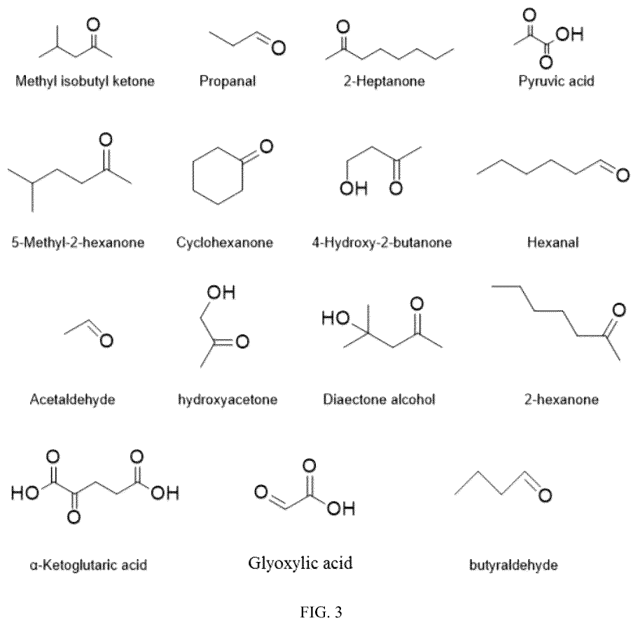

(1) plotting of acetophenone standard curve: preparing 8.3 uM, 16.6 uM, 24.9 uM, 33.2 uM, 41.6 uM, 83.2 uM and 124.8 uM standard acetophenone aqueous solutions respectively, mixing them evenly, adding them into a 1 mL quartz cuvette, and measuring their absorbance at the wavelength of 245 nm with an ultraviolet spectrophotometer to plot the standard curve of acetophenone concentration and its absorbance (y=0.008313x+0.01174, R2=0.9916);
(2) dissolving the pure enzyme obtained in Embodiment 3 on ice, where the final concentration of the pure enzyme is 8 mg/mL, adding 400 μL of the pure enzyme into 543 μL of Tris HCl buffer (pH 8.0, 0.1 M), and then adding 50 μL of pyridoxal phosphate PLP (10 mM) and 5 μL of phenylethylamine (40 mM, pH 8.0) in turn, preheating in a shaker (30° C., 1000 rpm) for 2 minutes, then adding 2 μL of 20 mM substrate as shown in FIG. 3, reacting for 2 hours, adding 100 μL of HCl (6 M) for quenching, then adding the same volume of n-butanol or ethyl acetate for extraction, centrifugally collecting the organic phase for drying, adding dried product into 1 mL Tris HCl buffer (pH 8.0, 0.1 M), diluting it 100 times, and measuring the absorbance of product acetophenone at the wavelength of 245 nm by ultraviolet spectrophotometer, and calculating the specific activity of transaminase according to the increase of acetophenone, reaction time and enzyme addition.

The formula for calculating the activity and specific activity of ω-transaminase is as follows: activity (U)=(A245 nm×dilution multiple-0.01174)/0.008313*time (h), where the specific activity A (U/g)=U/m (g) and m is the mass of pure enzyme involved in transamination reaction.

### Embodiment 5

### Determination of Recombinant ω-TA Substrate Spectrum

(1) dissolving the pure enzyme obtained in Embodiment 3 on ice, where the final concentration of the pure enzyme is 8 mg/mL, adding 400 μL of the pure enzyme into 543 μL of Tris HCl buffer (pH 8.0, 0.1 M), and then adding 50 μL of pyridoxal phosphate PLP (10 mM) and 5 μL of phenylethylamine (40 mM, pH8.0) in turn, preheating in a shaker (30° C., 100 rpm) for 2 minutes, then adding substrates with 20 mM each (methyl isobutyl ketone, propanal, 2-heptanone, pyruvic acid, 5-methyl-2-hexanone, cyclohexanone, 4-hydroxy-2-butanone, hexanal, acetaldehyde, hydroxyacetone, diaectone alcohol, 2-hexanone, α-ketoglutaric acid, glyoxylic acid and n-butyraldehyde) respectively shown in FIG. 3 for reacting for 2 hours;
(2) adding 100 μL HCl (6 M) for quenching, then adding the same volume of n-butanol or ethyl acetate for extraction, collecting the organic phase by centrifugation for drying, and diluting 100 times in 1 mL Tris HCl buffer (pH 8.0, 0.1 M);
(3) measuring the absorbance of acetophenone at the wavelength of 245 nm by ultraviolet spectrophotometer, and calculating the specific activity of transaminase according to the increase of acetophenone, reaction time and enzyme addition.

The treatment result of ω-transaminase of the present disclosure on substrates is shown in FIG. 3, and ω-transaminase has activity on the above substrates. The substrates are methyl isobutyl ketone, propanal, 2-heptanone, pyruvic acid, 5-methyl-2-hexanone, cyclohexanone, 4-hydroxy-2-butanone, hexanal, acetaldehyde, hydroxyacetone, diaectone alcohol, 2-Hexanone, α-ketoglutaric acid, glyoxylic acid and n-butyraldehyde.

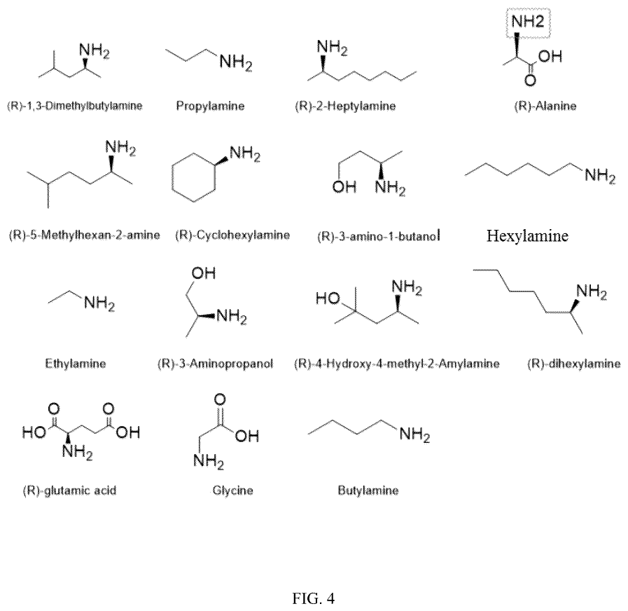

The chiral amino alcohols and chiral amines generated by the catalysis of the disclosure are shown in FIG. 4, and the chiral amino alcohols and chiral amines are respectively R-1,3-dimethylbutylamine, propylamine, R-2-heptylamine, R-alanine, R-5-methylhexyl-2 amine, R-cyclohexylamine, R-3-amino-1-butanol, hexylamine, ethylamine, R-3-aminopropanol, R-4-hydroxy-4-methyl-2-propylamine, R-dihexylamine, R-glutamic acid, glycine and butylamine.

According to the method described in Embodiment 4, the substrate spectrum of the recombinant ω-transaminase of the disclosure is broad, and it can catalyze the synthesis of 15 chiral amino alcohols and chiral amines with high added value, among which the catalytic activity for pyruvic acid is the highest, reaching 44785 U/g.

Especially, the specific activity of ω-transaminase provided by the disclosure to 4-hydroxy-2-butanone is 2397 U/g, which is much higher than the reported 596 U/g (Tang X, Zhang N, Ye G. et al. Efficient biosynthesis of (R)-3-amino-1-butanol by a novel (R)-selective transaminase from Actinobacteriasp. [J]. Journal of Biotechnology, 2019, 295:49-54.).

### Embodiment 6

### Determination of (R)-3-amino-1-butanol Catalyzed by Recombinant ω-TA by HPLC

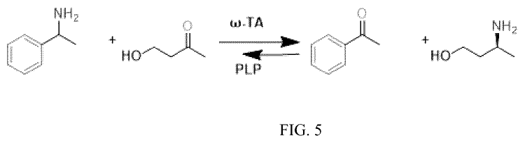

(1) using phenylethylamine as amino donor and 4-hydroxy-2-butanone as amino acceptor to generate (R)-3-amino-1-butanol and by-product acetophenone under the catalysis of ω-TA and coenzyme pyridoxal phosphate (PLP), as shown in FIG. 5;
(2) dissolving 10 μL of standard acetophenone and 10 μL of standard (R)-3-amino-1-butanol in 1 mL of acetonitrile to prepare 1% standard acetophenone sample and 1% standard (R)-3-amino-1-butanone sample, taking 1 mL of samples after catalytic reaction with ω-TA, and extracting with isoamyl acetate of the same volume for three times, drying, and adding 1 mL acetonitrile for dissolution;
(3) filtering the standard and reaction samples by 0.22 μm water-based filter membrane, followed by detecting by high performance liquid chromatography (HPLC) where the chromatographic column is Hypersil GOLD (5 μm, 250×4.6), the mobile phase is acetonitrile:water:triethanolamine=80:20:0.05, the flow rate is 1 mL/min, and the UV detection is carried out at 210 nm.

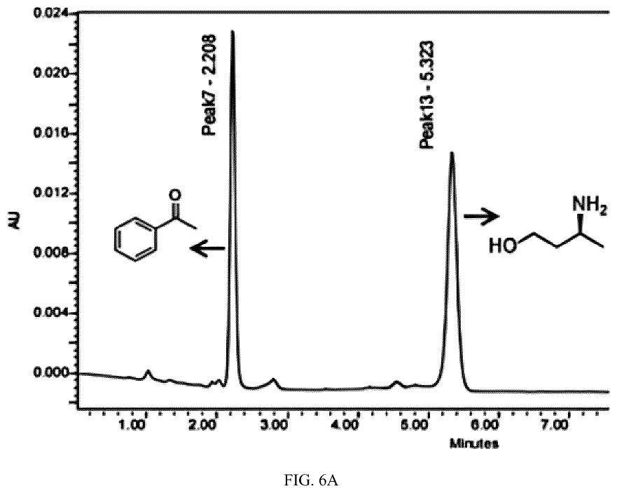

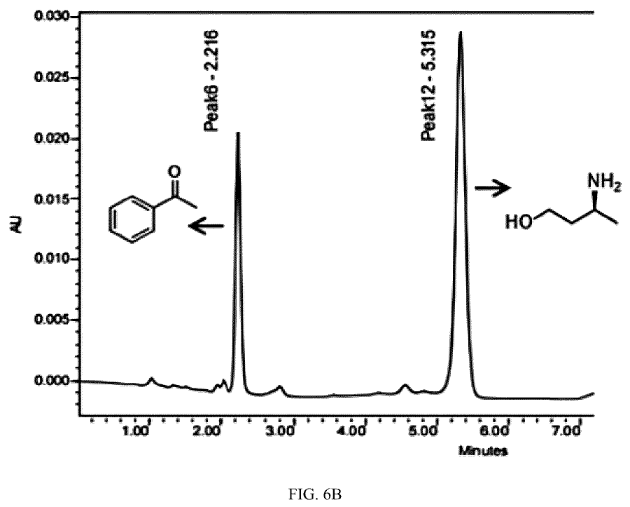

The standard result and experimental results are respectively shown in FIG. 6A and FIG. 6B. The results show that the retention time of standard acetophenone is 2.2 seconds, and that of standard (R)-3-amino-1-butanol is 5.2 seconds. The extracted samples have peaks at the same retention time, indicating that ω-TA catalyzed products form (R)-3-amino-1-butanol and by-products acetophenone.

### Embodiment 7

### Determination of Optimum Temperature for Catalytic Generation of (R)-3-amino-1-butanol by Recombinant ω-TA

The pure enzyme of recombinant ω-TA is taken out from the refrigerator at −80° C. and melted on ice. With phenylethylamine as the amino donor and 4-hydroxy-2-butanone as the amino acceptor, the activity of recombinant transaminase is determined at different reaction temperatures (25° C., 30° C., 35° C., 40° C., 45° C. and 50° C.), and other conditions remained unchanged.

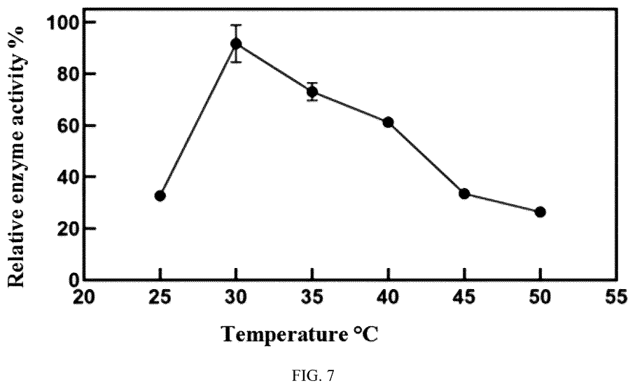

The results are shown in FIG. 7. When the reaction temperature is 30° C., the activity of (R)-3-amino-1-butanol which is generated by catalysis of ω-TA is the highest, that is, the optimal temperature for the catalytic production of (R)-3-amino-1-butanol by recombinant ω-TA is 30° C.

### Embodiment 8

### Determination of Optimum pH for Catalytic Generation of (R)-3-amino-1-butanol by Recombinant ω-TA

The pure enzyme of recombinant ω-TA is taken out from the refrigerator at −80° C. and melted on ice. With phenylethylamine as the amino donor and 4-hydroxy-2-butanone as the amino acceptor, according to the method described in Embodiment 4, the activity of recombinant transaminase is determined in different kinds of buffer solutions with different pH (disodium hydrogen phosphate-citric acid buffer solution with pH of 5.0, 5.5, 6.0, 6.5, 7.0; triethanolamine buffer solution with pH of 7.0, 7.5, 8.0, 8.5; Gly NaOH buffer solution with pH of 8.5, 9, 9.5, 10), and other conditions remained unchanged.

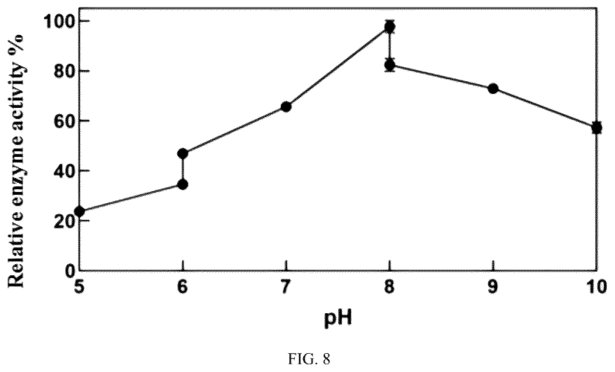

Results are shown in FIG. 8 and in the buffer solution of triethanolamine with pH of 8.0, ω-TA has the highest catalytic activity to produce (R)-3-amino-1-butanol, that is, the optimal pH of recombinant ω-TA is 8.0.

The above-mentioned embodiments only express specific cases of the disclosure, and their descriptions are more specific and detailed, but they should not be understood as limitations on the scope of present disclosure. It should be pointed out that for those skilled in the art, without departing from the concept of the disclosure, a number of variations and improvements can be made, which are within the scope of protection of the disclosure. Therefore, the scope of protection of this disclosure should be based on the appended claims.

1. SEQ ID NO: 1
ATGAGCGTCA ACAACCCGCA AACCCGTGAA TGGCAAACCC
TGAGCGGGGA GCATCACCTC GCACCCTTCA GTGACTACAA
GCAACTGAAG GAGAAGGGGC CGCGCATCAT CACCAAGGCC
CAGGGTGTGC ATTTGTGGGA TAGCGAGGGG CACAAGATCC
TCGACGGCAT GGCCGGTCTA TGGTGCGTGG CGGTCGGCTA
CGGCCGTGAA GAGCTGGTGC AGGCGGCAGA AAAACAGATG
CGCGAGCTGC CGTACTACAA CCTGTTCTTC CAGACCGCTC
ACCCGCCGGC GCTCGAGCTG GCCAAAGCGA TCACCGAAGT
GGCGCCGAAA GGTATGACCC ATGTGTTCTT CACCGGCTCC
GGCTCCGAAG GCAACGACAC TGTGCTGCGC ATGGTGCGTC
ACTACTGGGC GCTTAAGGGC AAACCGCACA AGCAGACCAT
CATCGGCCGT ATCAACGGTT ACCACGGTTC CACCTTCGCC
GGTGCTTGCC TGGGTGGCAT GAGCGGCATG CACGAGCAGG
GTGGCTTGCC GATCCCGGGT ATCGTTCACA TCCCTCAGCC
TTACTGGTTC GGCGAGGGCG GCGACATGAC CCCTGACGAA
TTCGGTGTCT GGGCTGCCGA GCAGCTGGAG AAGAAGATCC
TCGAAGTCGG CGAAGACAAC GTCGCTGCCT TCATCGCCGA
GCCGATCCAG GGCGCAGGTG GCGTGATCAT CCCGCCGGAA
ACCTACTGGC CCAAGGTGAA GGAGATCCTT GCCAAGTACG
ACATCCTGTT CGTCGCCGAC GAGGTGATCT GCGGCTTCGG
CCGTACCGGC GAGTGGTTCG GCTCTGACTA CTACGACCTC
AAGCCCGACC TGATGACCAT CGCGAAAGGC CTGACCTCTG
GTTACATCCC CATGGGCGGT GTGATCGTGC GTGACACCGT
GGCCAAGGTG ATCAGCGAAG GCGGCGACTT CAATCACGGT
TTCACCTACT CCGGCCACCC GGTGGCGGCC GCGGTGGGCC
TGGAAAACCT GCGCATCCTG CGTGACGAGA AAATTGTCGA
GAAGGCGCGC ACGGATACGG CACCGTATTT GCAAAAGCGT
TTGCGCGAGC TGCAGGACCA TCCTCTGGTG GGTGAAGTGC
GCGGCCTGGG CATGCTGGGT GCGATCGAGC TGGTCAAGGA
CAAGGCCACC CGCAGCCGTT ACGAAGGCAA AGGCGTGGGC
ATGATCTGTC GCACCTTCTG CTTTGAGAAC GGCCTGATCA
TGCGTGCGGT GGGTGACACC ATGATCATCG CGCCGCCGCT
GGTAATCAGC CATGCGGAGA TCGACGAACT GGTGGAAAAG
GCACGCAAGT GCCTGGATCT TACCCTTGAG GCGATTCGAT
AA
2. SEQ ID NO: 2
MSVNNPQTREWQTLSGEHHLAPFSDYKQLKEKGPRIITKAQGVHLWDSEG
HKILDGMAGLWCVAVGYGREELVQAAEKQMRELPYYNLFFQTAHPPALEL
AKAITEVAPKGMTHVFFTGSGSEGNDTVLRMVRHYWALKGKPHKQTIIGR
INGYHGSTFAGACLGGMSGMHEQGGLPIPGIVHIPQPYWFGEGGDMTPDE
FGVWAAEQLEKKILEVGEDNVAAFIAEPIQGAGGVIIPPETYWPKVKEIL
AKYDILFVADEVICGFGRTGEWFGSDYYDLKPDLMTIAKGLTSGYIPMGG
VIVRDTVAKVISEGGDFNHGFTYSGHPVAAAVGLENLRILRDEKIVEKAR
TDTAPYLQKRLRELQDHPLVGEVRGLGMLGAIELVKDKATRSRYEGKGVG
MICRTFCFENGLIMRAVGDTMIIAPPLVISHAEIDELVEKARKCLDLTLE
AIR

What is claimed is:

1. A Pseudomonas putida ω-transaminase gene with broad substrate spectrum, comprising a nucleotide sequence shown in SEQ ID NO: 1.

2. A protein ω-transaminase obtained by translating the ω-transaminase gene according to claim 1, comprising an amino acid sequence shown in SEQ ID NO: 2.

3. A method for preparing an enzyme preparation, comprising following steps: expressing a Pseudomonas putida ω-transaminase in a host cell to obtain a recombinant cell; lysing the recombinant cell to obtain crude enzyme solution, crude enzyme powder or pure enzyme; wherein an amino acid sequence of the Pseudomonas putida ω-transaminase gene is shown in SEQ ID NO: 2.

4. The method for preparing an enzyme preparation according to claim 3, wherein the recombinant cell contains a recombinant expression vector comprising the Pseudomonas putida ω-transaminase gene; a nucleotide sequence of the Pseudomonas putida ω-transaminase gene is shown in SEQ ID NO: 1.

5. The method for preparing an enzyme preparation according to claim 4, wherein the recombinant expression vector is any one of pET-28a, pET-29a, pET-30a, pET-32a, pCold I and pCold II.

6. The method for preparing an enzyme preparation according to claim 5, wherein the host cell is any one of BL21 (DE3), BL21 Star (DE3), BL21 (DE3) pLysS, C41 (DE3), C43 (DE3), BL21 (DE3) pLysE, Origami series, AD494 (DE3), BL21 Trxb (DE3), and SHuffle T7.
